(19) United States
(12) Patent Application Publication
(10) Pub. No.: US 2026/0248854 A1
(43) Pub. Date: Aug. 27, 2026

(54) TRANSFORMING GROWTH FACTOR-BETA SUPERFAMILY-IMPRINTED NATURAL KILLER CELLS FOR CANCER IMMUNOTHERAPY

(71) Applicant: RESEARCH INSTITUTE AT NATIONWIDE CHILDREN'S HOSPITAL, Columbus, OH (US)

(72) Inventors: Dean Anthony LEE, Canal Winchester, OH (US); Marcelo DE SOUZA FERNANDES PEREIRA, Columbus, OH (US)

(21) Appl. No.: 19/161,614

(22) PCT Filed: Mar. 4, 2024
(86) PCT No.: PCT/US2024/018354

§ 371 Date: Sep. 2, 2025

Related U.S. Application Data
(60) Provisional application No. 63/449,419, filed on Mar. 2, 2023.

(51) Int. Cl. A61K 35/17; A61K 39/00; A61K 40/15; A61P 35/04; C07K 16/28; C12N 5/0783

(52) U.S. Cl. CPC A61K 35/17; A61K 40/15; A61P 35/04; C07K 16/2896; C12N 5/0646; A61K 2039/505; C12N 2501/15

(57) ABSTRACT

The present disclosure relates to combination anti-cancer therapies using a transforming growth factor-beta (TGF-β) Superfamily-Imprinted Natural Killer (TGF-βi NK) cell and CD38-targeting agents and methods of use thereof.

### CROSS-REFERENCE TO RELATED APPLICATIONS

This PCT application claims priority to, and the benefit of, U.S. Provisional Patent Application No. 63/449,419, filed March 2^{nd}, 2023, entitled “TRANSFORMING GROWTH FACTOR-BETA SUPERFAMILY-IMPRINTED NATURAL KILLER CELLS FOR CANCER IMMUNOTHERAPY,” which is incorporated by reference herein in its entirety.

### FIELD

The present disclosure relates to combination anti-cancer therapies using a transforming growth factor-beta (TGF-β) Superfamily-Imprinted Natural Killer (TGF-βi NK) cell and CD38-targeting agents and methods of use thereof.

### BACKGROUND

Daratumumab (DARA) is a monoclonal antibody that binds CD38 and has been successfully tested for patients with CD38+ multiple myeloma (MM), and is under investigation for CD38+ T-ALL. DARA targets CD38+ cancer cells through several mechanisms, including antibody directed cell cytotoxicity (ADCC), which activates and leads NK cells to secrete cytotoxic granules and death ligands against the cancer targets. However, DARA also depletes NK cells, which highly express CD38. This is referred to as ‘fratricide,’ reducing the anti-tumor effectiveness of DARA. What are needed are new reagents and methods that can be used to preserve the killing action of NK cells in the presence of anti-CD38 antibody. The therapies and methods disclosed herein address these needs and more.

### SUMMARY

Disclosed are methods and compositions related to reducing CD38 on NK cells by educating (imprinting) NK cells with TGF-β and uses thereof in combination with CD38 inhibitors for treatment.

In one aspect, disclosed herein are methods of treating, inhibiting, reducing, decreasing, ameliorating, and/or preventing a cancer (such as, for example, leukemia, lymphoma, or myeloma) or an infectious disease in a subject in need thereof, comprising administering a therapeutically effective number of transforming growth factor-beta (TGF-β) Superfamily-Imprinted Natural Killer (TGF-βi NK) cells and a CD38-targeting agent (such as, for example, an anti-CD38 inhibitor such as an anti-CD38 antibody, including, but not limited to daratumumab or isatuximab) to the subject. In some aspects, the TGF-βi NK cells have a decreased expression of CD38. In some embodiments, the TGF-βi NK cells exhibit an increased resistance to a TGF-β superfamily cytokine (e.g., TGF-β). In some embodiments, the TGF-βi NK cells produced increased amounts of one or more of IFN-γ, TNF-α, and GM-CSF. In some embodiments, the TGF-βi NK cells show decreased levels of SMAD3 protein and/or TGFBR3 protein.

Also disclosed are methods of treating, inhibiting, reducing, decreasing, ameliorating, and/or preventing a cancer or an infectious disease of any preceding aspect, wherein the TGF-βi NK cells are prepared by incubating NK cells (e.g., in vitro or ex vivo) in the presence of TGF-β. In some aspects, the TGF-βi NK cells are prepared by incubating NK cells in the presence of feeder cells that have been engineered to express TGF-β or incubating NK cells in the presence of plasma membrane particles or exosomes derived from said feeder cells. In some embodiments, the feeder cells comprise PBMCs, RPMI8866, HFWT, K562 cells, EBV-LCL, NK cells (including, but not limited to PBMCs, RPMI 8866, NK-92, NK-92MI, NK-YTS, NK, NKL, KIL, KIL C.2, NK 3.3, NK-YS, HFWT, K562 cells) transfected with membrane bound IL-21, NK cells (including, but not limited to PBMCs, RPMI 8866, NK-92, NK-92MI, NK-YTS, NK, NKL, KIL, KIL C.2, NK 3.3, NK-YS, HFWT, K562 cells) transfected with membrane bound 4-1BBL, NK cells (including, but not limited to PBMCs, RPMI 8866, NK-92, NK-92MI, NK-YTS, NK, NKL, KIL, KIL C.2, NK 3.3, NK-YS, HFWT, K562 cells) transfected with membrane bound IL-15 and 4-1BBL, or NK cells (including, but not limited to PBMCs, RPMI 8866, NK-92, NK-92MI, NK-YTS, NK, NKL, KIL, KIL C.2, NK 3.3, NK-YS, HFWT, K562 cells) transfected with membrane bound IL-21 and/or 4-1BBL. In one aspect, the NK cells are incubated in the presence of the engineered feeder cells, plasma membrane particles, or exosomes for at least 7, 8, 9, 10, 11, 12, 13, 14, 15, 16, 17, 18, 19, 20, 21, 22, 23, 24, 25, 26, 27, 28, 29, 30, 45, or 60 days.

In one aspect, disclosed are methods of treating, inhibiting, reducing, decreasing, ameliorating, and/or preventing a cancer or an infectious disease of any preceding aspect, wherein the feeder cells further comprise at least one additional NK cell effector agent on its cell surface, wherein the at least one additional NK cell effector agent is a cytokine, an adhesion molecule, or an NK cell activating agent including, but not limited to 4-1BBL, IL-2, IL-12, IL-15, IL-18, IL-21, MICA, LFA-1, 2B4, CCR7, OX40L, UBLP2, BCM1/SLAMF2, NKG2D agonists, CD155, CD112, Jagged1, Jagged2, Delta-1, Pref-1, DNER, Jedi, SOM-11, wingless, CCN3, MAGP2, MAGP1, TSP2, YB-1, EGFL7, CCR7, DAP12, and DAP10, Notch ligands, NKp46 agonists, NKp44 agonists, NKp30 agonists, other NCR agonists, CD16 agonists. In one aspect, the at least one additional NK cell effector agent comprises IL-21, 4-1BBL, IL-15, IL-21 and 4-1BBL, IL-21 and IL-15, or IL-15 and 4-1BBL.

Also disclosed are methods of treating, inhibiting, reducing, decreasing, ameliorating, and/or preventing a cancer or an infectious disease of any preceding aspect, wherein the NK cells comprise memory-like NK cells such as NKG2C⁺, CD56^{bright} NK cells, CD56^{dim} NK cells, peripheral NK cells, NK T cells, or tumor infiltrating NK cells (including, but not limited to NK cells obtained from cell lines or obtained from a donor source. In some aspects, the NK cells are human NK cells or canine NK cells. In some aspects, the NK cells are primary NK cells (such as, for example NI cells obtained from a donor subject including, but not limited to an autologous donor, allogeneic donor, or syngeneic donor) or an NK cell line. In some aspects, the NK cells are activated in vitro or ex vivo.

In one aspect, disclosed herein are methods of treating, inhibiting, reducing, decreasing, ameliorating, and/or preventing a cancer or an infectious disease of any preceding aspect, wherein subject comprises tumor cells having increased levels of CD38 relative to a reference control.

Also disclosed herein are anti-cancer therapies comprising a transforming growth factor-beta (TGF-β) Superfamily-Imprinted Natural Killer (TGF-βi NK) cells and a CD38-targeting agent (such as, for example, an anti-CD38 inhibitor such as an anti-CD38 antibody, including, but not limited to daratumumab or isatuximab).

In one aspect, disclosed herein are anti-cancer therapies of any preceding aspect, wherein the TGF-βi NK cells are prepared by incubating NK cells (e.g., in vitro or ex vivo) in the presence of TGF-β. In some aspects, the TGF-βi NK cells are prepared by incubating NK cells in the presence of feeder cells that have been engineered to express TGF-β or incubating NK cells in the presence of plasma membrane particles or exosomes derived from said feeder cells. In some embodiments, the feeder cells comprise PBMCs, RPMI8866, HFWT, K562 cells, EBV-LCL, NK cells (including, but not limited to PBMCs, RPMI 8866, NK-92, NK-92MI, NK-YTS, NK, NKL, KIL, KIL C.2, NK 3.3, NK-YS, HFWT, K562 cells) transfected with membrane bound IL-21, NK cells (including, but not limited to PBMCs, RPMI 8866, NK-92, NK-92MI, NK-YTS, NK, NKL, KIL, KIL C.2, NK 3.3, NK-YS, HFWT, K562 cells) transfected with membrane bound 4-1BBL, NK cells (including, but not limited to PBMCs, RPMI 8866, NK-92, NK-92MI, NK-YTS, NK, NKL, KIL, KIL C.2, NK 3.3, NK-YS, HFWT, K562 cells) transfected with membrane bound IL-15 and 4-1BBL, or NK cells (including, but not limited to PBMCs, RPMI8866, NK-92, NK-92MI, NK-YTS, NK, NKL, KIL, KIL C.2, NK 3.3, NK-YS, HFWT, K 562 cells) transfected with membrane bound IL-21 and/or 4-1BBL. In one aspect, the NK cells are incubated in the presence of the engineered feeder cells, plasma membrane particles, or exosomes for at least 7, 8, 9, 10, 11, 12, 13, 14, 15, 16, 17, 18, 19, 20, 21, 22, 23, 24, 25, 26, 27, 28, 29, 30, 45, or 60 days.

Also disclosed herein are anti-cancer therapies of any preceding aspect, wherein the feeder cells further comprise at least one additional NK cell effector agent on its cell surface, wherein the at least one additional NK cell effector agent is a cytokine, an adhesion molecule, or an NK cell activating agent including, but not limited to 4-1BBL, IL-2, IL-12, IL-15, IL-18, IL-21, MICA, LFA-1, 2B4, CCR7, OX40L, UBLP2, BCM1/SLAMF2, NKG2D agonists, CD155, CD112, Jagged1, Jagged2, Delta-1, Pref-1, DNER, Jedi, SOM-11, wingless, CCN3, MAGP2, MAGP1, TSP2, YB-1, EGFL7, CCR7, DAP12, and DAP10, Notch ligands, NKp46 agonists, NKp44 agonists, NKp30 agonists, other NCR agonists, CD16 agonists. In one aspect, the at least one additional NK cell effector agent comprises IL-21, 4-1BBL, IL-15, IL-21 and 4-1BBL, IL-21 and IL-15, or IL-15 and 4-1BBL.

In one aspect, disclosed herein are anti-cancer therapies of any preceding aspect, wherein the NK cells comprise memory-like NK cells such as NKG2C⁺, CD56^{bright} NK cells, CD56^{dim} NK cells, peripheral NK cells, NK T cells, or tumor infiltrating NK cells (including, but not limited to NK cells obtained from cell lines or obtained from a donor source. In some aspects, the NK cells are human NK cells or canine NK cells. In some aspects, the NK cells are primary NK cells (such as, for example NI cells obtained from a donor subject including, but not limited to an autologous donor, allogeneic donor, or syngeneic donor) or an NK cell line. In some aspects, the NK cells are activated in vitro or ex vivo.

In one aspect, disclosed herein are modified natural killer (NK) cells, wherein the modified NK cells are cultured and/or prepared (e.g., in vitro or ex vivo) in the presence of TGF-β superfamily cytokine, and feeder cells that have been engineered to express TGF-β or plasma membrane particles or exosomes derived from said feeder cells for at least 7, 8, 9, 10, 11, 12, 13, 14, 15, 16, 17, 18, 19, 20, 21, 22, 23, 24, 25, 26, 27, 28, 29, 30, 45, or 60 days.

In some embodiments, the modified NK cell is prepared in the presence of feeder cells comprising PBMCs, RPMI8866, HFWT, K562 cells, EBV-LCL, NK cells (including, but not limited to PBMCs, RPMI 8866, NK-92, NK-92MI, NK-YTS, NK, NKL, KIL, KIL C.2, NK 3.3, NK-YS, HFWT, K562 cells) transfected with membrane bound IL-21, NK cells (including, but not limited to PBMCs, RPMI 8866, NK-92, NK-92MI, NK-YTS, NK, NKL, KIL, KIL C.2, NK 3.3, NK-YS, HFWT, K562 cells) transfected with membrane bound 4-1BBL, NK cells (including, but not limited to PBMCs, RPMI 8866, NK-92, NK-92MI, NK-YTS, NK, NKL, KIL, KIL C.2, NK 3.3, NK-YS, HFWT, K562 cells) transfected with membrane bound IL-15 and 4-1BBL, or NK cells (including, but not limited to PBMCs, RPMI8866, NK-92, NK-92MI, NK-YTS, NK, NKL, KIL, KIL C.2, NK 3.3, NK-YS, HFWT, K562 cells) transfected with membrane bound IL-21 and/or 4-1BBL. In some embodiments, the NK cell comprises increased resistance to TGF-β relative to a naturally occurring NK cell.

Also, disclosed herein, is a modified NK cell of any preceding aspect, wherein the feeder cells further comprise at least one additional NK cell effector agent on its cell surface, wherein the at least one additional NK cell effector agent is a cytokine, an adhesion molecule, or an NK cell activating agent including, but not limited to 4-1BBL, IL-2, IL-12, IL-15, IL-18, IL-21, MICA, LFA-1, 2B4, CCR7, OX40L, UBLP2, BCM1/SLAMF2, NKG2D agonists, CD155, CD112, Jagged1, Jagged2, Delta-1, Pref-1, DNER, Jedi, SOM-11, wingless, CCN3, MAGP2, MAGP1, TSP2, YB-1, EGFL7, CCR7, DAP12, and DAP10, Notch ligands, NKp46 agonists, NKp44 agonists, NKp30 agonists, other NCR agonists, CD16 agonists. In one aspect, the at least one additional NK cell effector agent comprises IL-21, 4-1BBL, IL-15, IL-21 and 4-1BBL, IL-21 and IL-15, or IL-15 and 4-1BBL.

In some embodiments, the modified NK cell of any preceding aspect is activated in vitro or ex vivo. In some embodiments, the modified NK cell of any preceding aspect is a human NK cell or a canine NK cell. In some embodiments, the modified NK cell of any preceding aspect is a primary NK cell or an NK cell line. In some embodiments, the modified NK cell of any preceding aspect comprises a memory-like NK cell such as NKG2C⁺CD56^{bright} NK cells, CD56^{dim} NK cells, peripheral NK cells, and NK T cells, or tumor infiltrating NK cells.

In some embodiments, the modified NK cell of any preceding aspect is obtained from a donor subject, including but not limited to an autologous donor, an allogeneic donor, and/or a syngeneic donor.

### BRIEF DESCRIPTION OF FIGURES

The accompanying figures, which are incorporated in and constitute a part of this specification, illustrate several aspects described below.

FIGS. 1A, 1B, 1C, and 1D show the real-time potency assessment for suspension target cells killed by WT and TGF-βi NK cells. The xCELLigence RTCA MP instrument (Agilent Technologies) was utilized for all impedance experiments. To adapt the xCELLigence E-Plate for analyzing killing of suspension target cells, the E-Plate wells were incubated with the tethering reagent CD29 for Kassumi or CD71 for K562 cell lines (AML and CML cell line respectively), at a concentration of 2 μg/mL, for 3 hours at 37° C. After wells were gently washed with PBS, tumor cells were seeded at a density of 60,000 cells per well. STD and TGF-βi NK cells cytotoxic activity were evaluated based on the viability of the tumor cells attached in E-Plate surface, as reflected by Cell Index values. Time course data of percentage of cell lysis of CML and AML (FIGS. 1A and 1C respectively). Graphical analysis of different time points (FIGS. 1B and 1D).

FIGS. 2A and 2B show the real-time potency assessment for adherent target cells killed by WT and TGF-βi NK cells. The xCELLigence RTCA MP instrument (Agilent Technologies) was utilized for all impedance experiments. tumor cells were seeded at a density of 20,000 cells per well. STD and TGF-βi NK cells cytotoxic activity were evaluated based on the viability of the tumor cells attached in E-Plate surface, as reflected by Cell Index values. Time course data of percentage of cell lysis of MEL-LM33 (FIGS. 2A and 2B represent 2:1 and 0.5:1 E:T ratio respectively).

FIGS. 3A, 3B, and 3C show that TGF-f imprinting downregulates CD38 and STAT1 signaling. (FIG. 3A) CD38 expression on STD and TGF-βi NK cells. CD38 expression was assessed by flow cytometry. Data showed by percentage and MFI. (FIG. 3B) RNAseq analysis. STAT1 expression and comparison between STD vs TGF-βi NK cells on FIG. 3C.

FIGS. 4A, 4B, 4C, 4D, 4E, and 4F show the favorable metabolic reprogramming of TGF-βi NK cells. FIGS. 4B and 4C show the mitochondrial and glycolytic stress, respectively. Summarized data of metabolic analysis of paired STD and TGF-βi NK cells (n=3; mean±SD). FIG. 4D shows the graphical analysis of Maximum respiratory capacity, and reserve capacity derived from FIG. 4B. FIG. 4E shows the Glycolysis, Glycolytic capacity and glycolytic reserve derived from FIG. 4C. FIG. 4F shows the ratio OCR/ECAR. All experiments were achieved using quintuplicate samples. FCCP, carbonyl cyanide-4-(trifluoromethoxy)phenylhydrazone; ROT/AA, rotenone and antimycin A. FIG. 4F also shows the Mito Tracker staining on STD and TGF-βi NK cells. Increase on Mito Tracker staining is associated with the metabolic shift from glycolysis to OXPHOS.

FIGS. 5A, 5B, and 5C show the resistance of TGF-βi NK cells to DARA-induced fratricide. Data of viability of STD and TGF-βi NK cells treated with DARA compared with that of control. Live cells and dead cells are shown in FIGS. 5A and 5B, respectively (n=1). FIG. 5C shows the representative data of changes in lysis with DARA of paired STD and TGF-βi NK cells against JURKAT, MOLT4 and RPMI8226 (T-ALL and Multiple myeloma cell lines, respectively).

FIGS. 6A, 6B, and 6C show the CD38 expression on expanded NK cells. FIG. 6A shows the representative FACs analysis of CD38+ NK cells percentage of CD38 expressing NK cells CD38 WT, TGF-βi or CD38KO. FIG. 6B shows the graph representative of FACs analysis. FIG. 6C shows the RNAseq analysis. Comparison between Naïve (nonexpanded NK cells) vs Expanded NK cells vs TGF-βI (TGF-β-imprinted).

FIGS. 7A, 7B, 7C, 7D, and 7E show the effects of TGF-β imprinting. FIG. 7A shows the enhanced production of pro-inflammatory cytokines. FIG. 7B shows the loss of sensitivity to TGF-β immunosuppression. FIG. 7C shows the downregulation of canonical SMAD3 signaling. FIG. 7D shows the reprogrammed tissue-resident addressin profile. FIG. 7E shows markedly improved serial killing.

FIGS. 8A, 8B, and 8C show that the TGF-β imprinting alters gene expression with 1,750 genes having a DESeqScore greater than 1.5.

FIG. 9 shows the TGF-β imprinting alters addressins gene expression (similar to FIG. 7D).

FIGS. 10A and 10B show that TGF-β imprinting improves tumor control.

FIG. 11 shows that TGF-β imprinting protects mice from liver metastasis.

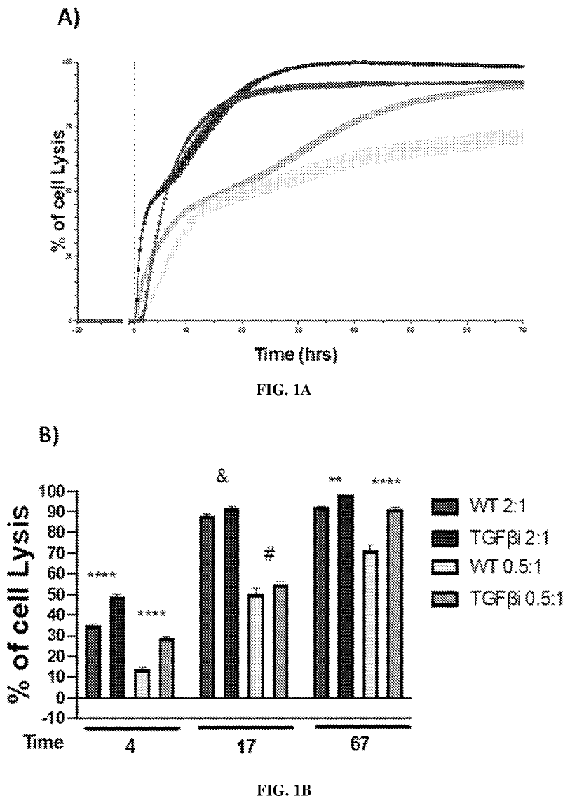

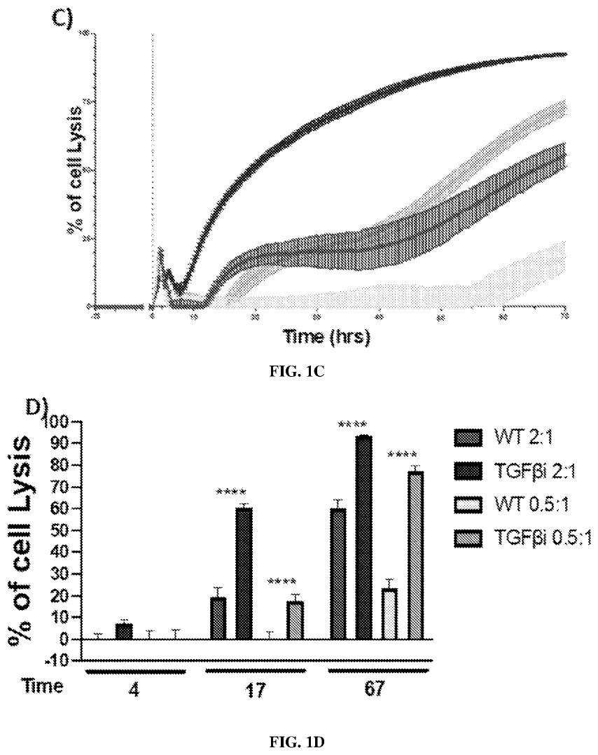

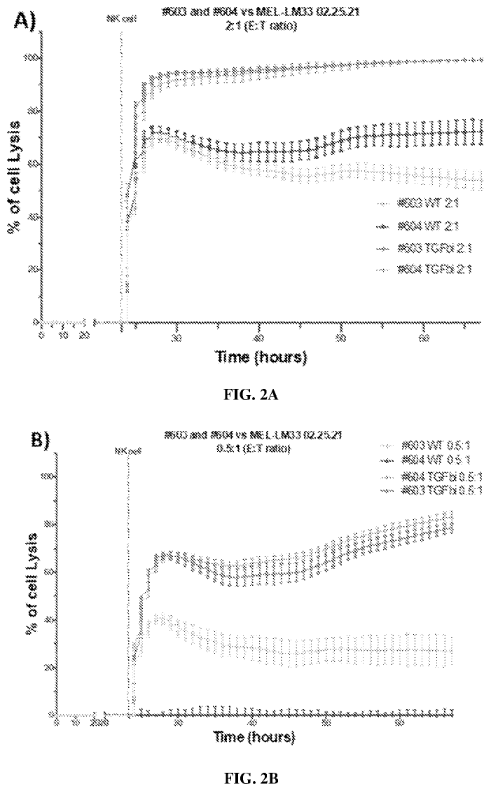

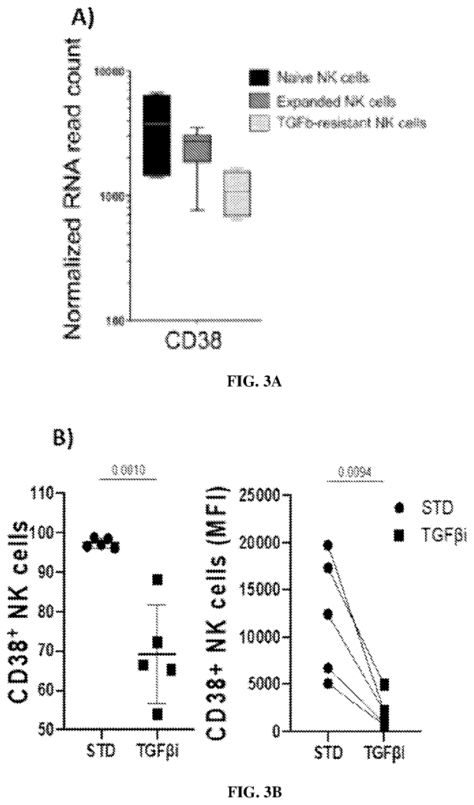

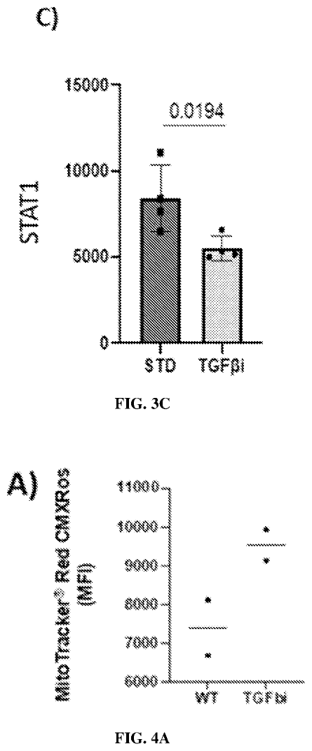

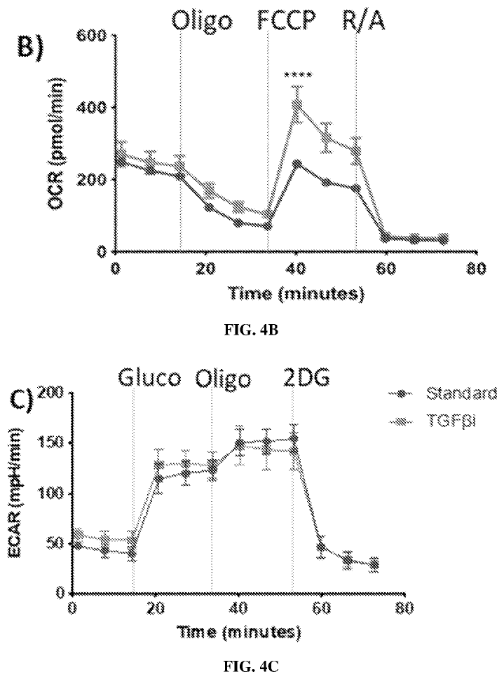

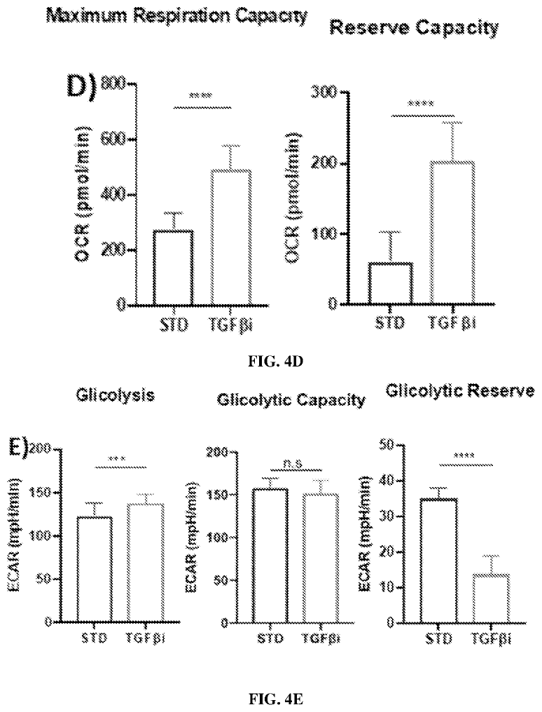

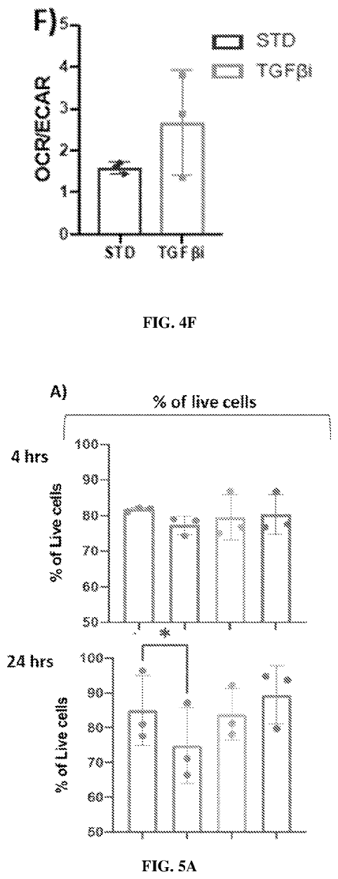

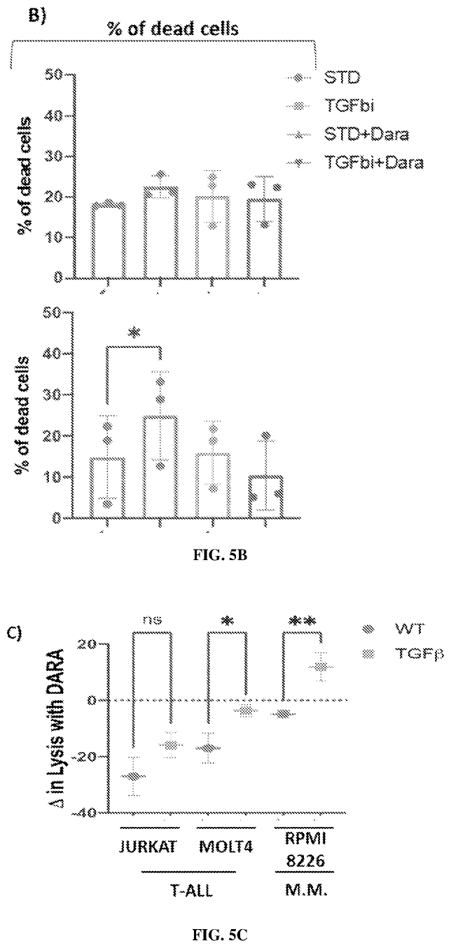

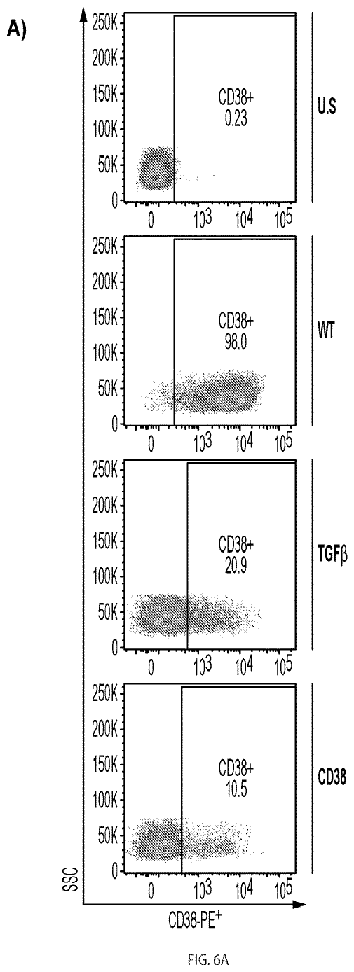

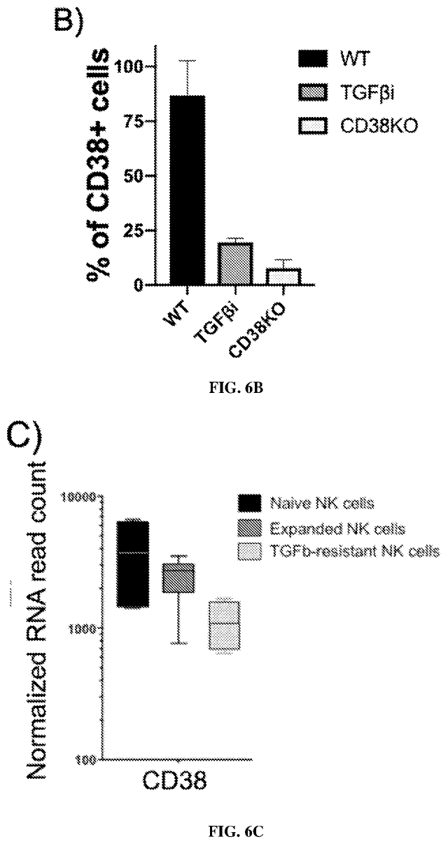

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd Ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As used herein, the terms “optional” or “optionally” mean that the subsequently described event or circumstance may or may not occur, and that the description includes instances where said event or circumstance occurs and instances where it does not.

As used herein, “N-terminal side” or “amino terminal end” refers to directionality of a peptide, polypeptide, or protein and may not mean the N-terminus. In some aspects, where a chimeric or fusion peptide, polypeptide, or protein is discussed, the N-terminal side may refer only to a component of the chimeric or fusion peptide, polypeptide, or protein and not the entire structure. For example, where a Fc domain is discussed, and the Fc domain is described as fused with its amino terminal end or N-terminal side facing intracellularly, contemplated herein are chimeric or fusion peptides, polypeptides, or proteins wherein the signal anchor is at the N-terminus of the chimeric or fusion construct and actually spans the cellular membrane. Thus, in such a chimera, the trans-membrane anchor is attached to the amino terminal side of the Fc domain, with the directionality of the Fc domain has the N-terminal side facing the cell which is inverted relative to an Fc domain on a typical B cell which would typically have the carboxy end spanning the cellular membrane and amino terminal end extending to the extracellular matrix.

The terms “peptide,” “polypeptide” and “protein” are used interchangeably to refer to a polymer of amino acid residues.

The term “sequence identity” as used herein, indicates a quantitative measure of the degree of identity between two sequences of substantially equal length. The percent identity of two sequences, whether nucleic acid or amino acid sequences, is the number of exact matches between two aligned sequences divided by the length of the shorter sequence and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found on the GenBank website. In general, the substitutions are conservative amino acid substitutions: limited to exchanges within members of group 1: glycine, alanine, valine, leucine, and Isoleucine; group 2: serine, cysteine, threonine, and methionine; group 3: proline; group 4: phenylalanine, tyrosine, and tryptophan; group 5: aspartate, glutamate, asparagine, and glutamine. Preferably, a percentage sequence identity is calculated over the whole length of sequences that are to be compared.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity.

As various changes could be made in the above-described cells and methods without departing from the scope of the invention, it is intended that all matter contained in the above description and in the examples given below, shall be interpreted as illustrative and not in a limiting sense.

An “increase” can refer to any change that results in a greater amount of a symptom, disease, composition, condition or activity. An increase can be any individual, median, or average increase in a condition, symptom, activity, composition in a statistically significant amount relative to a control. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount relative to a control. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration, but are undetectable or detectable at negligible amounts in distal parts of the subject's body. Administration includes self-administration and the administration by another.

“Treat,” “treating,” “treatment,” and grammatical variations thereof as used herein, include the administration of a composition with the intent or purpose of partially or completely preventing, delaying, curing, healing, alleviating, relieving, altering, remedying, ameliorating, improving, stabilizing, mitigating, and/or reducing the intensity or frequency of one or more a diseases or conditions, a symptom of a disease or condition, or an underlying cause of a disease or condition. Treatments according to the invention may be applied preventively, prophylactically, pallatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for day(s) to years prior to the manifestation of symptoms of a disease or an infection.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### Compositions and Methods of Treatment

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular TGF-β expressing engineered feeder cell is disclosed and discussed and a number of modifications that can be made to a number of molecules including the TGF-β expressing engineered feeder cell are discussed, specifically contemplated is each and every combination and permutation of TGF-β expressing engineered feeder cell and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

In one aspect, disclosed herein a method of treating cancer or an infectious disease in a subject in need thereof, comprising administering a therapeutically effective number of transforming growth factor-beta (TGF-β) Superfamily-Imprinted Natural Killer (TGF-βi NK) cells and a CD38-targeting agent to the subject.

Also disclosed herein are anti-cancer therapies comprising a transforming growth factor-beta (TGF-β) Superfamily-Imprinted Natural Killer (TGF-βi NK) cell and a CD38-targeting agent (e.g., a CD38 inhibitor).

In some embodiments, the TGF-βi NK cells are prepared by incubating NK cells (e.g., in vitro or ex vivo) in the presence of TGF-β, in the presence of feeder cells that have been engineered to express TGF-β, or incubating NK cells in the presence of plasma membrane particles or exosomes derived from said feeder cells.

In some embodiments, the feeder cells comprise PBMCs, RPMI8866, HFWT, K562 cells, EBV-LCL, NK cells (including, but not limited to PBMCs, RPMI8866, NK-92, NK-92MI, NK-YTS, NK, NKL, KIL, KIL C.2, NK3.3, NK-YS, HFWT, K562 cells) transfected with membrane bound IL-21, NK cells (including, but not limited to PBMCs, RPMI8866, NK-92, NK-92MI, NK-YTS, NK, NKL, KIL, KIL C.2, NK3.3, NK-YS, HFWT, K562 cells) transfected with membrane bound 4-1BBL, NK cells (including, but not limited to PBMCs, RPMI8866, NK-92, NK-92MI, NK-YTS, NK, NKL, KIL, KIL C.2, NK3.3, NK-YS, HFWT, K562 cells) transfected with membrane bound IL-15 and 4-1BBL, or NK cells (including, but not limited to PBMCs, RPMI8866, NK-92, NK-92MI, NK-YTS, NK, NKL, KIL, KIL C.2, NK3.3, NK-YS, HFWT, K562 cells) transfected with membrane bound IL-21 and/or 4-1BBL. In one aspect, the NK cells are incubated in the presence of the engineered feeder cells, plasma membrane particles, or exosomes for at least 7, 8, 9, 10, 11, 12, 13, 14, 15, 16, 17, 18, 19, 20, 21, 22, 23, 24, 25, 26, 27, 28, 29, 30, 45, or 60 days.

The NK cells incubated by the methods disclosed herein can be primary NK cells or an NK cell line. In some embodiments, the NK cells comprise memory-like NK cells such as NKG2C⁺, CD56^{bright} NK cells, CD56^{dim} NK cells, peripheral NK cells, NK T cells, or tumor infiltrating NK cells (including, but not limited to NK cells obtained from cell lines or obtained from a donor source (such as for example, an autologous donor, allogeneic donor, or syngeneic donor).

In some aspects, disclosed herein are methods of treating, inhibiting, reducing, decreasing, ameliorating, and/or preventing a cancer or an infectious disease in a subject in need thereof as well as anti-cancer therapies, wherein the TGF-βi NK cells exhibit an increased resistance to a TGF-superfamily cytokine (e.g., TGF-β). In some embodiments, the TGF-βi NK cells produced increased amounts of one or more of IFN-γ, TNF-α, and GM-CSF. In some embodiments, the TGF-βi NK cells show decreased levels of SMAD3 protein and/or TGFBR3 protein.

In some aspects, the NK cells are human NK cells or canine NK cells.

In some aspects, the NK cells are primary NK cells (such as, for example NI cells obtained from a donor subject including, but not limited to an autologous donor, allogeneic donor, or syngeneic donor) or an NK cell line.

In some aspects, the NK cells are activated in vitro or ex vivo.

Also disclosed herein are anti-cancer therapies, wherein the feeder cells further comprise at least one additional NK cell effector agent on its cell surface, wherein the at least one additional NK cell effector agent is a cytokine, an adhesion molecule, or an NK cell activating agent including, but not limited to 4-1BBL, IL-2, IL-12, IL-15, IL-18, IL-21, MICA, LFA-1, 2B4, CCR7, OX40L, UBLP2, BCM1/SLAMF2, NKG2D agonists, CD155, CD112, Jagged1, Jagged2, Delta-1, Pref-1, DNER, Jedi, SOM-11, wingless, CCN3, MAGP2, MAGP1, TSP2, YB-1, EGFL7, CCR7, DAP12, and DAP10, Notch ligands, NKp46 agonists, NKp44 agonists, NKp30 agonists, other NCR agonists, CD16 agonists. In one aspect, the at least one additional NK cell effector agent comprises IL-21, 4-1BBL, IL-15, IL-21 and 4-1BBL, IL-21 and IL-15, or IL-15 and 4-1BBL.

In one aspect, disclosed herein are modified natural killer (NK) cells, wherein the modified K cells are cultured and/or prepared (e.g., in vitro or ex vivo) in the presence of TGF-β superfamily cytokine, and feeder cells that have been engineered to express TGF-β or plasma membrane particles or exosomes derived from said feeder cells for at least 7, 8, 9, 10, 11, 12, 13, 14, 15, 16, 17, 18, 19, 20, 21, 22, 23, 24, 25, 26, 27, 28, 29, 30, 45, or 60 days.

In some embodiments, the modified NK cells are prepared in the presence of feeder cells comprising PBMCs, RPMI8866, HFWT, K562 cells, EBV-LCL, NK cells (including, but not limited to PBMCs, RPMI 8866, NK-92, NK-92MI, NK-YTS, NK, NKL, KIL, KIL C.2, NK3.3, NK-YS, HFWT, K562 cells) transfected with membrane bound IL-21, NK cells (including, but not limited to PBMCs, RPMI 8866, NK-92, NK-92MI, NK-YTS, NK, NKL, KIL, KIL C.2, NK3.3, NK-YS, HFWT, K562 cells) transfected with membrane bound 4-1BBL, NK cells (including, but not limited to PBMCs, RPMI 8866, NK-92, NK-92MI, NK-YTS, NK, NKL, KIL, KIL C.2, NK3.3, NK-YS, HFWT, K562 cells) transfected with membrane bound IL-15 and 4-1BBL, or NK cells (including, but not limited to PBMCs, RPMI8866, NK-92, NK-92MI, NK-YTS, NK, NKL, KIL, KIL C.2, NK 3.3, NK-YS, HFWT, K562 cells) transfected with membrane bound IL-21 and/or 4-1BBL. In some embodiments, the NK cell comprises increased resistance to TGF-β relative to a naturally occurring NK cell.

Also, disclosed herein, is a modified NK cells, wherein the feeder cells further comprise at least one additional NK cell effector agent on its cell surface, wherein the at least one additional NK cell effector agent is a cytokine, an adhesion molecule, or an NK cell activating agent including, but not limited to 4-1BBL, IL-2, IL-12, IL-15, IL-18, IL-21, MICA, LFA-1, 2B4, CCR7, OX40L, UBLP2, BCM1/SLAMF2, NKG2D agonists, CD155, CD112, Jagged1, Jagged2, Delta-1, Pref-1, DNER, Jedi, SOM-11, wingless, CCN3, MAGP2, MAGP1, TSP2, YB-1, EGFL7, CCR7, DAP12, and DAP10, Notch ligands, NKp46 agonists, NKp44 agonists, NKp30 agonists, other NCR agonists, CD16 agonists. In one aspect, the at least one additional NK cell effector agent comprises IL-21, 4-1BBL, IL-15, IL-21 and 4-1BBL, IL-21 and IL-15, or IL-15 and 4-1BBL.

In some embodiments, the modified NK cell is activated in vitro or ex vivo.

In some embodiments, the modified NK cell is a human NK cell or a canine NK cell. In some embodiments, the modified NK cell of any preceding aspect is a primary NK cell or an NK cell line.

In some embodiments, the modified NK cell of
comprises a memory-like NK cell such as NKG2C⁺CD56^{bright} NK cells, CD56^{dim} NK cells, peripheral NK cells, and NK T cells, or tumor infiltrating NK cells.

In some embodiments, the modified NK cell of any preceding aspect is obtained from a donor subject, including but not limited to an autologous donor, an allogeneic donor, and/or a syngeneic donor.

In some embodiments, the CD38-targeting agent used in the disclosed anti-cancer therapies and methods treating, inhibiting, reducing, decreasing, ameliorating, and/or preventing a cancer or infectious disease is an anti-CD38 antibody. The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with CD38. The antibodies can be tested for their desired activity using the in vitro assays described herein, or by analogous methods, after which their in vivo therapeutic and/or prophylactic activities are tested according to known clinical testing methods. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody or fragments thereof” encompasses chimeric antibodies and hybrid antibodies, with dual or multiple antigen or epitope specificities, and fragments, such as F(ab′)2, Fab′, Fab, Fv, sFv, scFv, and the like, including hybrid fragments. Thus, fragments of the antibodies that retain the ability to bind their specific antigens are provided. For example, fragments of antibodies which maintain CD38 binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)). Accordingly, in some embodiments, the anti-CD38 antibody comprises a fragment crystallizable region (Fc region) binds to an Fc receptor or lacks a Fc region.

Also included within the meaning of “antibody or fragments thereof” are conjugates of antibody fragments and antigen binding proteins (single chain antibodies).

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

In some embodiments, the anti-CD38 antibody is daratumumab or isatuximab. The TGF-βi NK cells can be administered prior to, concurrently with, and/or following the administration of the CD38-targeting agent (e.g., a CD38 inhibitor).

### a) TGF-β Imprinted Natural Killer Cells

In some aspects, disclosed herein is a natural killer (NK) cell or NK cell line cultured in the presence of a TGF-β superfamily cytokine, referred to herein as TGF-βi NK cells. This includes NK cells or a cell line produced by the methods described herein, and compositions comprising the NK cells provided herein. In a particular aspect, the composition is a pharmaceutical composition comprising one or more of the NK cells or cell lines provided herein. In some embodiments, the TGF-βi NK cells exhibit increased resistance to TGF-β.

The TGF-βi NK cells can be allogenic or autologous cells. In some aspects, the NK cell is a mammalian NK cell. Examples of “mammalian” or “mammals” include primates (e.g., human), canines, felines, rodents, porcine, ruminants, and the like. Specific examples include humans, dogs, cats, horses, cows, sheep, goats, rabbits, guinea pigs, rats and mice. In a particular embodiment, the mammalian NK cell is a human NK cell.

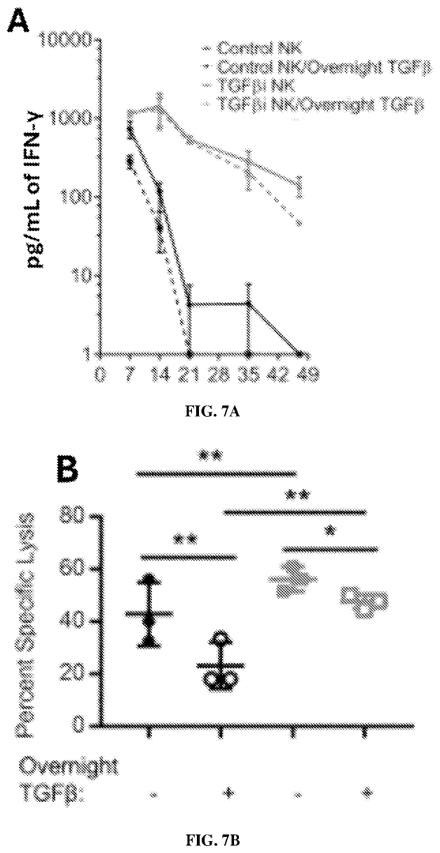

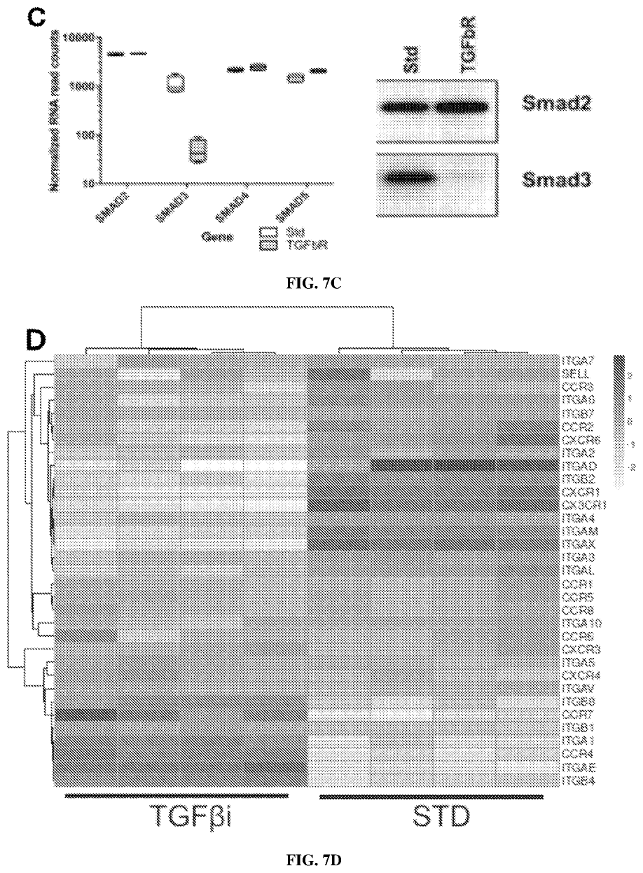

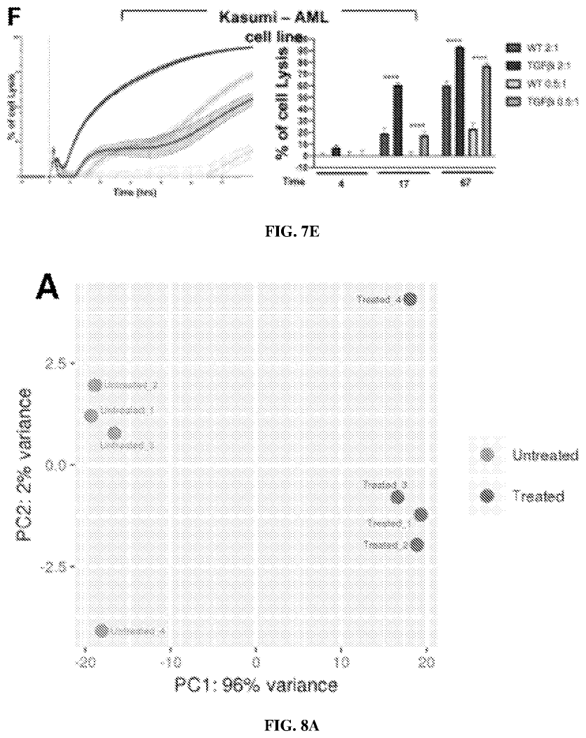

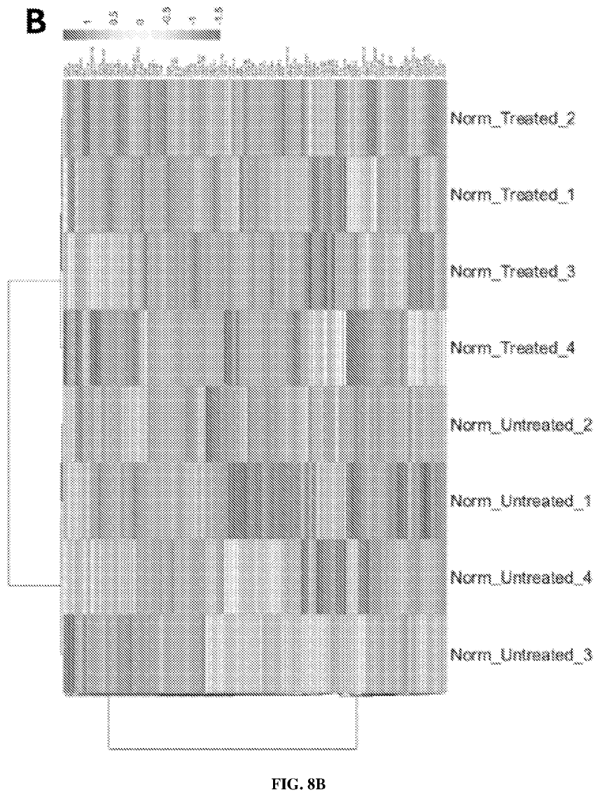

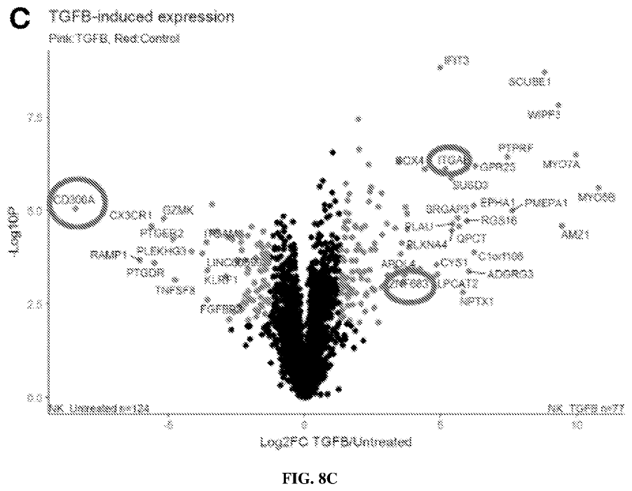

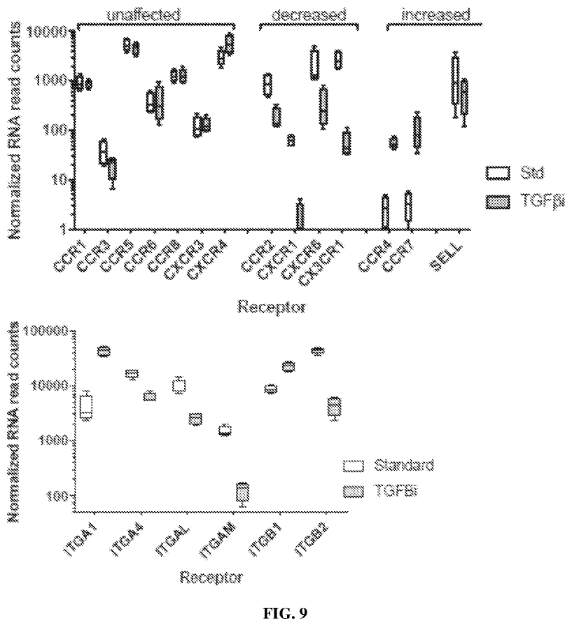

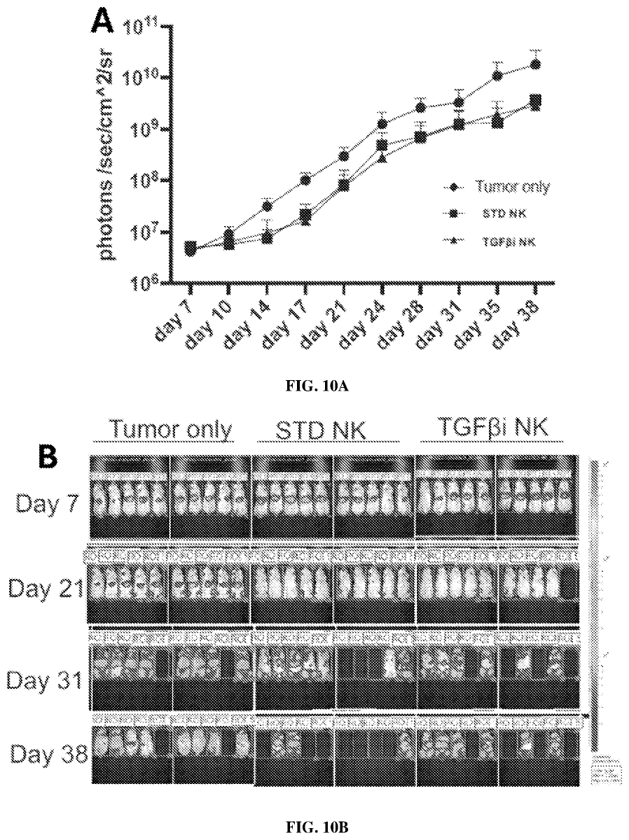

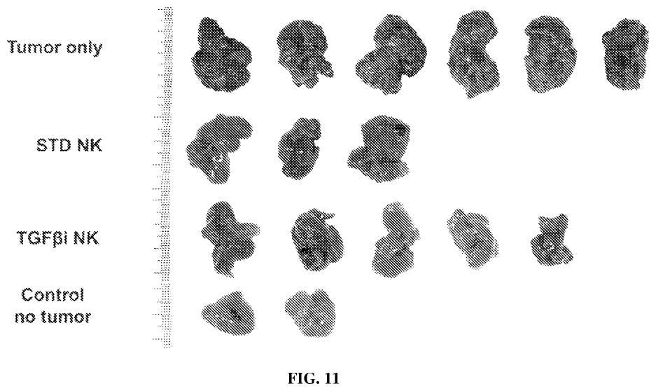

The TGF-βi NK cells exhibit a number of characteristics that distinguish them from naturally occurring NK cells. In some embodiments, the NK cell or cell lines exhibit increased resistance to TGF-β. In other embodiments, the NK cells produce and increased amount of interferon-γ (IFN-γ), and/or tumor necrosis factor-α (TNF-α), and/or Granulocyte-macrophage colony-stimulating factor (GM-CSF). In further embodiments, the NK cells show decreased levels of SMAD family member 3 (SMAD3) protein and/or Transforming growth factor beta receptor III (TGFBR3) protein. SMAD proteins received their name as a contraction of the names of the C. elegans Sma and Drosophila Mad (Derynck et al., Cell, 95(6), p 737-740, 1998) and are transcriptional activators of TGF-β responses. The TGF-βi NK cells exhibit a number of characteristics that distinguish them from naturally occurring NK cells. In some embodiments, the NK cells have a gene expression profile substantially similar to that shown in FIG. 7D. A gene expression profile that is substantially similar is one in which the gene expression is within 10% of that shown. In some embodiments, the TGF-βi NK cells produce increased amounts of one or more of IFN-γ, TNF-α and GM-CSF protein. In some embodiments, the NK cell or cell lines exhibit increased expression of SCUBE1, MYO7A, KLF3, WIPF3, and EPHA1.

The TGF-βi NK cells exhibit a number of characteristics that distinguish them from naturally occurring NK cells. In some embodiments, the TGF-βi NK cells show decreased levels of SMAD3 protein and/or TGFBR3 protein and/or CD38 protein. In some embodiments, the NK cell or cell lines exhibit decreased expression of CD300A, SGSM1, SMAD3, TBX21, and GZMK, TGFBR3, and GZMA.

In one aspect, disclosed herein are methods of treating, inhibiting, reducing, decreasing, ameliorating, and/or preventing a cancer and/or metastasis, wherein the NK cells are incubated in the presence of the engineered feeder cells, plasma membrane particles, or exosomes for at least 7, 8, 9, 10, 11, 12, 13, 14, 15, 16, 17, 18, 19, 20, 21, 22, 23, 24, 25, 26, 27, 28, 29, 30, 45, or 60 days.

The disclosed compositions can be used to treat any disease where uncontrolled cellular proliferation occurs such as cancers. A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer.

In some aspects, disclosed herein are methods, compositions, uses and/or anti-cancer therapy of any preceding aspects, wherein cancer cells have increased levels of CD38 in comparison to a reference level.

### b) Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, PA 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid-or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 g/kg to up to 100 mg/kg of body weight or more per day, depending on the factors mentioned above.

### c) Methods of Making TGF-β Imprinted NK Cells

As noted throughout the primary purpose of the TGF-β imprinting is to make NK cells that have decreased expression of CD38. Accordingly, in one aspect, disclosed herein are methods of generating TGF-β imprinted NK cells, comprising incubating NK cells in the presence of the engineered feeder cells, plasma membrane particles, or exosome disclosed herein. For example, disclosed herein are methods of generating TGF-β imprinted NK cells, comprising incubating NK cells in the presence of feeder cells (including but not limited to PBMCs, RPMI8866, HFWT, K562 cells, EBV-LCL, NK cells (including, but not limited to NK-92, NK-92MI, NK-YTS, NK, NKL, KIL, KIL C.2, NK3.3, NK-YS) transfected with membrane bound IL-21, NK cells (including, but not limited to NK-92, NK-92MI, NK-YTS, NK, NKL, KIL, KIL C.2, NK3.3, NK-YS) transfected with membrane bound 4-1BBL, NK cells (including, but not limited to NK-92, NK-92MI, NK-YTS, NK, NKL, KIL, KIL C.2, NK3.3, NK-YS) transfected with membrane bound IL-15 and 4-1BBL, or NK cells (including, but not limited to NK-92, NK-92MI, NK-YTS, NK, NKL, KIL, KIL C.2, NK 3.3, NK-YS) transfected with membrane bound IL-21 and 4-1BBL) that have been engineered to express TGF-β or incubating NK cells in the presence of plasma membrane particles or exosomes derived from said feeder cells. Preferably, the TGF-β expressed by the feeder cells is membrane bound.

In one aspect, disclosed herein are methods of generating TGF-β imprinted NK cells wherein the feeder cells comprise at least one additional NK cell effector agent on its cell surface, wherein the at least one additional NK cell effector agent is a cytokine, an adhesion molecule, or an NK cell activating agent (such as, for example, NK cell effector agents including, but not limited to 4-1BBL, IL-2, IL-12, IL-15, IL-18, IL-21, MICA, LFA-1, 2B4, CCR7, OX40L, UBLP2, BCM1/SLAMF2, NKG2D agonists, CD155, CD112, Jagged1, Jagged2, Delta-1, Pref-1, DNER, Jedi, SOM-11, wingless, CCN3, MAGP2, MAGP1, TSP2, YB-1, EGFL7, CCR7, DAP12, and DAP10, Notch ligands, NKp46 agonists, NKp44 agonists, NKp30 agonists, other NCR agonists, CD16 agonists). In one aspect the at least one additional NK cell effector agent comprises IL-21, 4-1BBL, IL-15, IL-21 and 4-1BBL, IL-21 and IL-15, or IL-15 and 4-1BBL. Preferably, the at least one additional NK cell effector agent is a membrane bound NK cell effector agent.

It is understood and herein contemplated that the methods of generating TGF-β imprinted NK cells disclosed herein can be used on any NK cell (exogenous or endogenous) where resistance TGF-β is desired. Accordingly, disclosed herein are methods of generating TGF-β imprinted NK cells wherein the NK cells comprise memory-like NK cells such as NKG2C⁺, CD56^{bright} NK cells, CD56^{dim} NK cells, peripheral NK cells, NK T cells, or tumor infiltrating NK cells (including, but not limited to NK cells obtained from cell lines or obtained from a donor source (such as for example, an autologous donor, allogeneic donor, or syngeneic donor).

To generate TGF-β imprinted NK cells the NK cells must be exposed to the feeder cell, exosome, or plasma membrane particle expressing TGF-β (on its membrane or soluble), or in the presence of soluble TGF-β, for a period of time to confer resistance. Thus, in one aspect, disclosed herein are methods of generating TGF-β imprinted NK cells, wherein the NK cells are incubated in the presence of TGF-β and the engineered feeder cells, plasma membrane particles, or exosomes for at least 7, 8, 9, 10, 11, 12, 13, 14, 15, 16, 17, 18, 19, 20, 21, 22, 23, 24, 25, 26, 27, 28, 29, 30, 45, or 60 days. It is further understood and herein contemplated that the NK cells can be cultured for additional periods of time after exposure to the engineered feeder cells or plasma membrane particles or exosomes derived from said feeder cells. In one aspect, the NK cells can be contacted with the engineered feeder cells or plasma membrane particles or exosomes derived from said feeder cells for between 7 and 21 days, preferably between 7 and 14 days.

As noted throughout, the disclosed methods generate NK cells that are TGF-β imprinted. Thus, in one aspect, disclosed herein are TGF-β imprinted NK cells made by the method of generating TGF-β imprinted NK cells disclosed herein.

In some instances, the plasma membrane particles or exosome derived from the engineered feeder cells can be obtained by nitrogen cavitation.

In general, the cell is maintained under conditions appropriate for cell growth and/or maintenance. Suitable cell culture conditions are well known in the art and are described, for example, in Santiago et al., Proc. Natl. Acad. Sci. USA, 2008, 105:5809-5814; Moehle et al. Proc. Natl. Acad. Sci. USA, 2007, 104:3055-3060; Urnov et al., Nature, 2005, 435:646-651; and Lombardo et al., Nat. Biotechnol., 2007, 25:1298-1306. Those of skill in the art appreciate that methods for culturing cells are known in the art and can and will vary depending on the cell type. Routine optimization may be used, in all cases, to determine the best techniques for a particular cell type.

### d) Dosage and Administration

The TGF-βi NK cells should be administered and dosed in accordance with good medical practice, taking into account the site and method of administration, scheduling of administration, patient age, sex, body weight, the nature and severity of the disorder to be treated or prevented, and other factors known to medical practitioners. The cells may be administered in a single dose or in divided doses. The pharmaceutically “effective amount” for purposes herein is thus determined by such considerations as are known in the art. The amount must be effective to achieve improvement, including but not limited to improved survival rate or more rapid recovery, or improvement or elimination of symptoms and other indicators as are selected as appropriate measures by those skilled in the art.

Typically said dose is about 10×10⁶ cells/kg of subject weight or lower, is about 9×10⁶ cells/kg or lower, is about 8×10⁶ cells/kg or lower, is about 7×10⁶ cells/kg or lower, is about 6×10⁶ cells/kg or lower, is about 5×10⁶ cells/kg or lower. In an alternative embodiment said dose may be between about 0.25×10⁶ cells/kg to about 5×10⁶ cells/kg; or more preferably about 1×10⁶ cells/kg to about 5×10⁶ cells/kg. Accordingly in further alternative embodiments the dose may be about 0.25×10⁶ cells/kg, 0.5×10⁶ cells/kg, 0.6×10⁶ cells/kg, 0.7×10⁶ cells/kg; 0.8×10⁶ cells/kg; 0.9×10⁶ cells/kg; 1.1×10⁶ cells/kg; 1.2×10⁶ cells/kg; 1.3×10⁶ cells/kg; 1.4×10⁶ cells/kg; 1.5×10⁶ cells/kg; 1.6×10⁶ cells/kg; 1.7×10⁶ cells/kg; 1.8×10⁶ cells/kg; 1.9×10⁶ cells/kg or 2×10⁶ cells/kg. The dose may, in other embodiments, be between 0.1 and 1 million cells/kg; or between 1 and 2 million cells/kg; or between 2 and 3 million cells/kg; or between 3 and 4 million cells/kg; or between 4 and 5 million cells/kg; or between 5 and 6 million cells/kg; or between 6 and 7 million cells/kg; or between 7 and 8 million cells/kg; or between 8 and 9 million cells/kg; or between 9 and 10 million cells/kg.

A cancer can be selected from, but is not limited to, a hematologic cancer, lymphoma, colorectal cancer, colon cancer, lung cancer, a head and neck cancer, ovarian cancer, prostate cancer, testicular cancer, renal cancer, skin cancer, cervical cancer, pancreatic cancer, and breast cancer. In one aspect, the cancer comprises a solid tumor. In another aspect, the cancer is selected from acute myeloid leukemia, myelodysplastic syndrome, chronic myeloid leukemia, acute lymphoblastic leukemia, myelofibrosis, multiple myeloma. In another aspect, the cancer is selected from a leukemia, a lymphoma, a sarcoma, a carcinoma and may originate in the marrow, brain, lung, breast, pancreas, liver, head and neck, skin, reproductive tract, prostate, colon, liver, kidney, intraperitoneum, bone, joint, and eye.

It is intended herein that the disclosed methods of inhibiting, reducing, and/or preventing cancer metastasis and/or recurrence can comprise the administration of any anti-cancer agent known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), AsparaginaseErwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (AsparaginaseErwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human,, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). Also contemplated herein are chemotherapeutics that are PD1/PDL1 blockade inhibitors (such as, for example, lambrolizumab, nivolumab, pembrolizumab, pidilizumab, BMS-936559, Atezolizumab, Durvalumab, or Avelumab). It is also intended herein that the disclosed uses of the disclosed compositions and/or an engineered NK cell population for inhibiting, reducing, and/or preventing cancer metastasis and/or recurrence can comprise use in combination the use of any anti-cancer agent known in the art including, but not limited to those agents listed above.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Cd38^{low} Natural Killer (NK) Cells for Cancer Immunotherapy

Daratumumab (DARA) is a monoclonal antibody that binds CD38 and has been successfully tested for patients with CD38+ multiple myeloma (MM), and is under investigation for CD38+ T-ALL. DARA targets CD38+ cancer cells through several mechanisms, including antibody directed cell cytotoxicity (ADCC), which activates and leads NK cells to secrete cytotoxic granules and death ligands against the cancer targets. However, DARA also depletes NK cells, which highly express CD38. This is referred to as ‘fratricide,’reducing the anti-tumor effectiveness of DARA.

Naeimi-Kararoudi et al., showed that CRISPR/Cas9 system was very effective to delete CD38 in ex vivo expanded peripheral blood NK cells. These CD38KO NK cells were completely resistant to DARA-induced fratricide, showing superior persistence in immune deficient mice pretreated with DARA, and enhanced ADCC activity against CD38-expressing MM cell lines and primary MM cells.

The application of CRISPR-based gene modification to the clinic is difficult, but TGFβ-imprinting may be a viable non-genetic alternative if it yields equal or better results, and thus may support development of a clinical trial for MM patients of a novel cellular therapeutic product—TGF-βi-CD38^{low}NK cells—in combination with DARA to treat MM.

### Example 2: TGF-βi—Imprinting of Primary NK Cells Decreasing CD38-Nadase, Promotes a Tissue-Resident Addressin Profile, and Improves Cytotoxicity and Metabolism

Transforming growth factor-beta (TGFβ) is a potent immunosuppressive cytokine that inhibits the anti-tumor responses of NK cells and T cells. However, the stimulation of natural killer (NK) cells with pro-inflammatory cytokines decreases NK cellsensitivity to TGFβ. Previously, it was demonstrated that TGFβ-imprinting (TGFβi) during IL-21-driven expansion further decreases NK cell sensitivity to TGFβ through SMAD3suppression and enhances a pro-inflammatory phenotype with hypersecretion of IFN-γ, TNF-α, and GM-CSF.

To evaluate serial killing ability of TGFβi-NK cells a Real-Time Cell Analysis was performed. TGFβi NK cells showed faster and more efficient cytotoxicity against both liquid(K562 and Kasumi) and solid (MEL33 and MEL41) cancer cell lines when compared to standard expanded NK cells. Additionally, TGF-βi-NK cells had significantly better killing than standard expanded NK cells at low E:T ratio (0.5:1). To better understand the impact of TGFβ-imprinting on gene expression in NK cells, RNA-seq was performed. At the mRNA level, TGF-βi-imprinting alters chemokine receptor expression, decreasing CCR2, CXCR1, CXCR6, and CX3CR1 and increasing CCR4 and 7. In addition, TGF-β-imprinted NK cells showed an increase in integrins like ITGA1, ITGB1, and ITGAE showing that TGF-β-imprinting was able to induce reprogrammed tissue-resident addressin profile similar to that of ILC1s. Next, the efficacy of TGF-βi NK cells was evaluated in a disseminated tumor model, which showed that Standard and TGF-βi NK cells were similar in their ability to control the primary tumor, but TGF-βi NK cells were superior in protecting the mice from liver metastasis (FIG. 7).

RNAseq also revealed that TGF-β imprinting significantly suppresses CD38 expression in NK cells, which was by flow cytometry. As CD38 is an ectoenzyme that regulates NAD+, a critical component of OXPHOS in both T and NK cells, the effect of TGF-βi on NK cell metabolism was examined. Higher oxygen consumption rates (OCR) were observed with higher OCR/ECAR ratios in TGF-βi NK cells than Standard NK cells. Having markedly decreased levels of surface CD38 expression, the ability to resist fratricide mediated by daratumumab (DARA) was also evaluated. TGF-βi NK cells are found to be resistant to DARA-induced fratricide and have higher cytotoxicity against CD38+ cell lines in presence of DARA. FIG. 7.

Herein, it is shown that TGFβi suppresses CD38 resulting in improved OXPHOS and elimination of fratricide, and increases NK cell potency, tissue homing, and serial killing. TGF-βi-NK cells combined with CD38 targeting is considered for CD38+malignancies.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

1. A method of treating cancer or an infectious disease in a subject in need thereof, comprising administering a therapeutically effective number of transforming growth factor-beta (TGF-β) Superfamily-Imprinted Natural Killer (TGF-βi NK) cells and a CD38-targeting agent to the subject.

2. The method of claim 1, wherein the TGF-βi NK cells have a decreased expression of CD38.

3. The method of claim 1, wherein the TGF-βi NK cells have an increased resistance to a TGF-β superfamily cytokine.

4. The method of claim 1, wherein the TGF-βi NK cells have increased resistance to TGF-β.

5. The method of claim 1, wherein the TGF-βi NK cells produced increased amounts of one or more of IFN-γ, TNF-α, and GM-CSF.

6. The method of claim 1, wherein the TGF-βi NK cells show decreased levels of SMAD3 protein and/or TGFBR3 protein.

7. (canceled)

8. The method of claim 1, wherein the TGF-βi NK cells are prepared by incubating a NK cells cell in the presence of feeder cells that have been engineered to express TGF-β or incubating the NK cell in the presence of plasma membrane particles or exosomes derived from said feeder cells.

9. The method of claim 8, wherein the feeder cells comprise PBMCs, RPMI8866, HFWT, K562 cells, EBV-LCL, NK cells transfected with membrane bound IL-21, NK cells transfected with membrane bound 4-1BBL, NK cells transfected with membrane bound IL-15 and 4-1BBL, or NK cells transfected with membrane bound IL-21 and 4-1BBL.

10. The method of claim 8, wherein the feeder cells further comprise at least one additional NK cell effector agent on its cell surface, wherein the at least one additional NK cell effector agent is a cytokine, an adhesion molecule, or an NK cell activating agent.

11. The method of claim 10, wherein the at least one additional NK cell effector agent is selected from 4-1BBL, IL- 2, IL-12, IL-15, IL- 18, IL-21, MICA, LFA-1, 2B 4, CCR 7, OX40L, UBLP2, BCM1/SLAMF2, NKG2D agonists, CD155, CD112, Jagged1, Jagged2, Delta-1, Pref-1, DNER, Jedi, SOM-11, wingless, CCN3, MAGP2, MAGP1, TSP2, YB-1, EGFL7, CCR7, DAP12, and DAP10, Notch ligands, NKp46 agonists, NKp44 agonists, NKp30 agonists, other NCR agonists, CD16 agonists.

12. (canceled)

13. The method of claim 8, wherein the NK cell is activated in vitro or ex vivo.

14. The method of claim 8, wherein the NK cell is human NK cells or canine NK cells.

15. The method of claim 8, wherein the NK cell is primary NK cells or an NK cell line.

16. The method of claim 8, wherein the cell comprises a NKG2C⁺CD56^{bright} NK cell, a CD56^{dim} NK cell, a peripheral NK cell, a NK T cell, or a tumor infiltrating NK cell.

17. (canceled)

18. The method of claim 8, wherein the NK cell is obtained from autologous donor, allogeneic donor, or syngeneic donor.

19. The method of claim 8, wherein the NK cell is incubated in the presence of the engineered feeder cells, plasma membrane particles, or exosomes for at least 7, 8, 9, 10, 11, 12, 13, 14, 15, 16, 17, 18, 19, 20, 21, 22, 23, 24, 25, 26, 27, 28, 29, 30, 45, or 60 days.

20. The method of claim 1, wherein the CD38-targeting agent is a CD38 inhibitor or an anti-CD38 antibody.

21. (canceled)

22. (canceled)

23. The method of claim 20, wherein the anti-CD38 antibody is daratumumab or isatuximab.

24-26. (canceled)

27. An anti-cancer therapy comprising a transforming growth factor-beta (TGF-β) Superfamily-Imprinted Natural Killer (TGF-βi NK) cell and a CD38-targeting agent.

28-42. (canceled)

43. A modified natural killer (NK) cell, wherein the modified NK cell is prepared by culturing a first NK cell in the presence of a TGF-β superfamily cytokine and incubated in the presence of feeder cells that have been engineered to express TGF-β or incubating in the presence of plasma membrane particles or exosomes derived from said feeder cells.

44-55. (canceled)
